(19) United States
(12) Patent Application Publication
(10) Pub. No.: US 2009/0018343 A1
(43) Pub. Date: Jan. 15, 2009

(54) Fluorous Oxazolidinone Chiral Auxiliary Compounds and Methods of Manufacture

(75) Inventors: Philip Gregory Hultin, Winnipeg (CA); Jason Ellis Hein, Winnipeg (CA)

Correspondence Address: Ade & Company Inc., 2157 Henderson HWY., Winnipeg,, MB, R2G 1P9, CA

(73) Assignee: The University of Manitoba, Winnipeg ,MB (CA)

(21) Appl. No.: 11/886,417

(22) PCT Filed: Mar. 16, 2006
(86) PCT No.: PCT/CA2006/000409

§ 371 Date: Jun. 26, 2008

Related U.S. Application Data
(60) Provisional application No. 60/661,914, filed on Mar. 16, 2005.

(51) Int. Cl. C07D 263/22

(52) U.S. Cl. 548/229

(57) ABSTRACT

The present invention relates generally to perfluoroalkyl chiral auxiliary compounds and methods of manufacture. These compounds have the functionality to effectively support the synthesis of chiral compounds in single reactions, high-throughput parallel reactions, or combinatorial reactions The invention relates to two oxazolidinone chiral auxiliaries (1) and (2): wherein R_{f} is a perfluoroalkyl group having the general formula (CH₂)ₓ—C_{y}F_{2y+1} where x=1-5 and y=4-10 and wherein B is an unfunctionalized aryl, alkyl or arylalkyl group in a preferred embodiment, x=2 and y=6 and B is derived from unfunctionalized amino acids The amino acids may be from either the D- or L-series, and are preferably enantiomerically pure or in very high enantiomeric excess in either configuration.

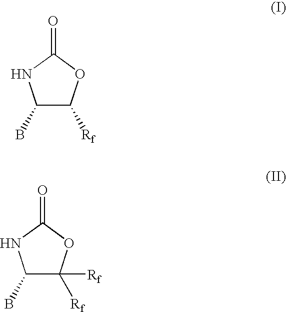

### FIELD OF THE INVENTION

The present invention relates generally to perfluoroalkyl chiral auxiliary compounds and methods of manufacture.

### BACKGROUND OF THE INVENTION

High-throughput synthetic methods are becoming important in pharmaceutical discovery. Many high-throughput syntheses employ supported techniques to facilitate recovery of the products. Asymmetric synthesis in a high-throughput environment would be greatly assisted by combining chiral auxiliary methodologies with supported synthetic techniques. The benefits from this approach are twofold. First, supported materials can be rapidly separated from excess and/or spent reagents and other reaction debris, allowing chiral adducts, as well as the valuable chiral auxiliary to be easily recovered. Second, supported materials can easily be applied to automated synthetic techniques, making the auxiliary an important tool for asymmetric induction in high-throughput chemistry.

Chiral auxiliaries using standard insoluble polymer supports have been synthesized, however, the heterogeneous nature of this system can lead to numerous complications.¹ Typically, the yield and/or selectivity of the supported reactions are very different from the solution phase analogues.² Furthermore, monitoring the progress of a reaction or fine-tuning the reaction conditions is not trivial, as characterization of reaction materials often requires cleavage from the support. Soluble polymeric supports, such as non-cross-linked polystyrene (NCPS) and poly(ethylene glycol), have also been functionalized with chiral materials and used in a number of different asymmetric reactions. However, harsh reaction conditions and strong Lewis acids can damage the support, leading to loss of the supported auxiliary and poor yield or selectivity upon subsequent reactions with the recovered material. There are several examples of particular systems which function extremely well,^{3, 4, 5} however, these are not general and cannot be applied to a broad variety of reactions.

Ultimately, there is a need for a new kind of support for asymmetric chemistry. This support should provide facile and selective recovery of supported material yet should be insensitive to a wide variety of reaction conditions. Most importantly, it must be chemically innocuous, and not interfere with the formation of the discrete complexes necessary for highly stereoselective reactions.

Fluorous methods are a powerful alternative to many polymer-supported approaches and meet the outlined criteria. Since 1997, Curran et al.^{6, 7, 8, 9, 10} have led the development of fluorous tags as soluble supports for organic synthesis, applying them to many organic transformations. They have not, however, explored the use of fluorous-tagged chiral auxiliaries.

Oxazolidinone auxiliaries derived from inexpensive amino acid starting materials are versatile compounds, which can be used in numerous C-C bond-forming reactions.¹¹ Supported oxazolidinones have shown promise, but a single supported auxiliary has yet to be made that can match the efficiency and selectivity of the standard, unsupported oxazolidinone auxiliaries.

It is, therefore, desirable to provide a supported oxazolidinone chiral auxiliary having the functionality to effectively support the synthesis of chiral compounds in single reactions, high-throughput parallel reactions, or combinatorial reactions.

### SUMMARY OF THE INVENTION

In accordance with the invention, there is provided a compound of the formula

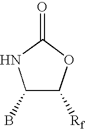

wherein R_{f} is a perfluroalkyl group having the general formula (CH2)ₓ—C_{y}F_{2y+1} where x=1-5 and y=4-10 and wherein B is an unfunctionalized aryl, arylaykyl, or alkyl group.

In a further embodiment, the invention provides a compound of the formula

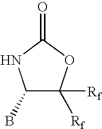

wherein R_{f} is a perfluroalkyl group having the general formula (CH2)ₓ—C_{y}F_{2y+1} where x=1-5 and y=4-10 and wherein B is an unfunctionalized aryl, arylalkyl, or alkyl group.

In preferred embodiments, the compound are enantomerically pure and x=2 and y=6.

In one embodiment, B is derived from unfunctionalized amino acids.

In another embodiment, the invention provides a method of synthesizing the above compounds comprising the steps of:
synthesizing an N-carbamate protected acyl species from an unfunctionalized amino acid;
subjecting the N-carbamate protected acyl species to a perfluoroalkyl lithium addition reaction using a perfluoroalkyllithium species to form a (perfluoroalkyl)ketone;
subjecting the (perfluoroalkyl)ketone to a diastereoselective reduction reaction to form a protected amino alcohol having an anti configuration;
subjecting the protected amino alcohol to a cyclization reaction to form the compound of claim 1 or claim 2.
In one embodiment of the method the N-carbamate protected acyl species is an N-(ethoxycarbonyl) group, and B is CH₂(C₆H₅).

In another embodiment, t-BuLi is used to form the perfluoroalkyllithium species, in a mixture of diethyl ether and hexanes or other hydrocarbon co-solvent at a temperature not exceeding −60° C.

In a still further embodiment, the N-carbamoyl acyl species is first deprotonated using a sacrificial base.

In yet another embodiment, the sacrificial base is selected from any one of or a combination of n-BuLi, s-BuLi, t-BuLi, NaH, KH or other alkali metal hydride.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 is a schematic flow diagram of the method in accordance with the invention;

FIG. 2 includes examples of asymmetric transformations demonstrating the proven utility of the new compound;

FIG. 3 is a synthetic scheme detailing the multi-step synthesis of a natural product in accordance with one embodiment of the invention;

FIG. 4 is a synthetic scheme detailing the multi-step synthesis of a natural product in accordance with one embodiment of the invention;

FIG. 5 is a process diagram detailing the practical application of the new material in the aforementioned syntheses.

### DETAILED DESCRIPTION

### Oxazolidinones

Two new oxazolidinone chiral auxiliaries (1) and (2) have been synthesized:

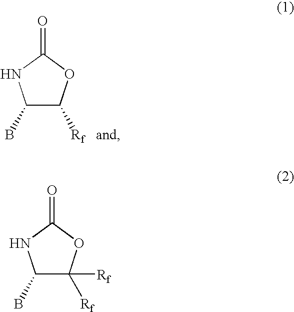

wherein R_{f} is a perfluoroalkyl group having the general formula (CH₂)ₓ—C_{y}F_{2y+1} where x=1-5 and y=4-10 and wherein B is an unfunctionalized aryl, alkyl or arylalkyl group. In a preferred embodiment, x=2 and y=6 and B is derived from unfunctionalized amino acids. The amino acids may be from either the D- or L-series, and are preferably enantiomerically pure or in very high enantiomeric excess in either configuration.

### Generalized Procedure

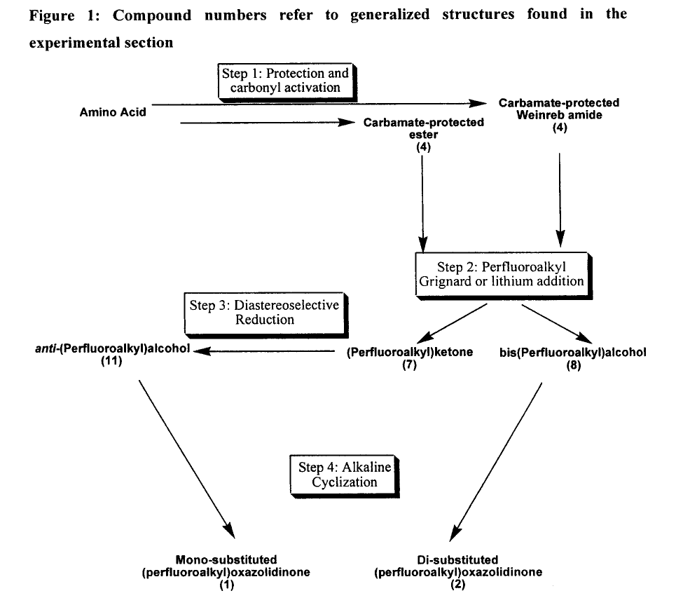

Compounds 1 and 2 are synthesized by a generalized process described below and with reference to FIG. 1.

### Step 1—Synthesis of N-Protected Electrophile

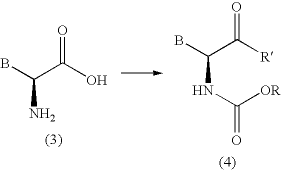

where R is alkyl or aryl and R′ is a leaving group moiety such as O-alkyl, O-aryl, N(alkyl)₂, or N(alkyl)(Oalkyl).

A carbamate-protected ester (4) is synthesized from an unfunctionalized amino acid (3) such as phenylalanine, valine, leucine or isoleucine utilizing N-protection and carboxylic acid activation methods. Representative examples of the synthesis of N-protected esters are as follows:

### Methyl (S)-N-(benzyloxycarbonyl)-phenylalaninate

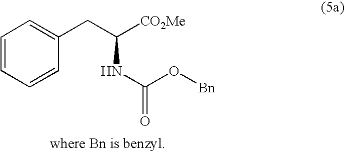

_{L}-Phenylalanine (5 g, 30.25 mmol) was suspended in THF (500 mL) and refluxed for 1 h to produce a concentrated solution. While the solution was still at reflux benzyl chloroformate (4.3 mL, 30.25 mmol) was added producing a milky solution. The mixture was refluxed for an additional 2 hrs, after which the reaction was cooled to room temperature and filtered to remove unreacted L-phenylalanine present as a fine white precipitate. The filtrate was concentrated under vacuum to produce a clear oil, which solidified when triturated with petroleum ether. The solid was filtered to give 7.0 g (77%) of N-CBz phenylalanine as a white solid. The ¹H and ¹³C NMR matched literature values.¹² allowing the material to be carried on without further purification.

N-CBz phenylalanine (5 g, 16.7 mmol) was dissolved in methanol (100 mL) and cooled to 0° C. Boron trifluoride etherate (4.44 mL, 35.07 mmol) was added dropwise over a period of 15 min. The reaction mixture was then removed from the ice bath and heated to reflux for 1 h before cooling and removing the solvent using a rotary evaporator. The crude residue was suspended with ice water and extracted with ethyl acetate (3×125 mL). The combined ether extracts was washed with 1 M NaHCO₃, and brine, then was dried with MgSO₄ and evaporated to dryness to give (4.5 g, 86%) of methyl (S)-N-(benzyloxycarbonyl)-phenylalaninate as a clear oil whose ¹H and ¹³C NMR spectra matched literature values.¹³ After drying the material thoroughly under high vacuum it was carried through without further purification.

### Methyl (S)-N-(isopropoxycarbonyl)-phenylalaninate

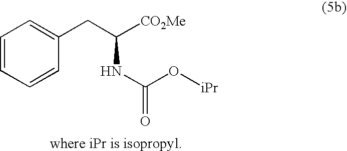

MeOH (250 mL) was added to _{L}-phenylalanine (15 g, 90.8 mmol) and the mixture was cooled to 0° C. Thionyl chloride (13.2 mL, 181.6 mmol) was added dropwise over a period of 45 min, during which time the solution cleared and began to boil gently. The reaction mixture was then refluxed for 4 h before cooling and removing the solvent using a rotary evaporator. This produced a crude white solid, which was resuspended and evaporated thrice with 100 mL portions of methanol to remove excess HCl. The product was then triturated with 150 mL of hexane and filtered, yielding 19.2g (˜99%) of methyl phenylalaninate hydrochloride as a white powder whose ¹H and ¹³C NMR spectra matched literature values.¹⁴ After drying the material under high vacuum it was carried through without further purification.

Methyl phenylalaninate hydrochloride (20 g, 92.8 mmol) was suspended in THF (100 mL) and cooled to 0° C. Triethylamine (27.83 mL, 199.5 mmol) was added dropwise, during which time the solution cleared. Isopropyl chloroformate (102.08 mL of a 1M solution in hexane, 102.08 mmol) was then added dropwise and the solution was allowed to stir at 0° C. for 2 hrs. After this time the reaction mixture was concentrated using a rotary evaporator, and the resulting residue was dissolved in ethyl acetate (400 mL) and water (100 mL). The organic fraction was separated and washed successively with 1M HCl, 1M NaHCO₃, and brine. The organic fraction was then dried with MgSO₄ and evaporated to give a crude white solid. The material was recrystallized from petroleum ether to produce (14.43 g, 70%) of methyl (S)-N-(isopropoxycarbonyl)-phenylalaninate as a cottony white material whose ¹H and ¹³C NMR spectra matched literature values.¹⁵

### Methyl (S)-N-(ethoxycarbonyl)-phenylalaninate

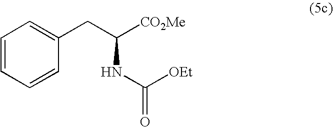

MeOH (250 mL) was added to L-phenylalanine (15 g, 90.8 mmol) and the mixture was cooled to 0° C. Thionyl chloride (13.2 mL, 181.6 mmol) was added dropwise over a period of 45 min, during which time the solution cleared and began to boil gently. The reaction mixture was then refluxed for 4 h before cooling and removing the solvent using a rotary evaporator. This produced a crude white solid, which was resuspended and evaporated thrice with 100 mL portions of methanol to remove excess HCl. The product was then triturated with 150 mL of hexane and filtered, yielding 19.2g (˜99%) of methyl phenylalaninate hydrochloride as a white powder whose ¹H and ¹³C NMR spectra matched literature values.¹⁴ After drying the material under high vacuum it was carried through without further purification.

Methyl phenylalaninate hydrochloride (10 g, 46.4 mmol) was suspended in THF (100 mL) and cooled to 0° C. Triethylamine (27.83 mL, 199.5 mmol) was added dropwise, during which time the solution cleared. Ethyl chloroformate (4.863 mL, 51.04 mmol) was then added dropwise and the solution was allowed warm to room temperature and then was stirred for 2 hrs. After this time the reaction mixture was concentrated using a rotary evaporator, and the resulting residue was dissolved in ethyl acetate (200 mL) and water (100 mL). The organic fraction was separated and washed successively with 1M HCl, 1M NaHCO₃, and brine. The organic fraction was then dried with MgSO₄ and evaporated to give a crude oil. The material was eluted through a 10 cm pad of silica gel with 4:1 Hex:EtOAc to produce (10.14 g, 87%) of methyl (S)-N-(ethoxycarbonyl)-phenylalaninate as a clear oil whose ¹H and ¹³C NMR spectra matched literature values.¹⁶

An alternate methodology (Step 1A) synthesized carbamate-protected Weinreb amides for subsequent perfluroroalkyl addition. Representative examples are described below.

_{L}-Phenylalanine (20 g, 0.12 mol) was suspended in water (100 mL) and methanol (100 mL). NaHCO₃ (˜16 g) was added, followed after 10 min by the alkyl chloroformate (1.3 eqv, 0.157 mol) producing a clear solution. The mixture was then stirred at high speed overnight. The alkaline solution was extracted with ethyl ether (2×100 mL) and the organic phase was discarded. The aqueous phase was then acidified with 2M HCl and extracted with ethyl ether (3×125 ml). The combined ether extracts were dried with MgSO₄ and evaporated to dryness. This produced 22.85 g (˜98%) of a clear oil. This material could be triturated with petroleum ether or hexanes to give the appropriate N-protected phenylalanine carbamate as a white solid.

The solid N-carbamoyl phenylalanine (0.11 mol) was then dissolved in THF (200 mL) and cooled to −30° C. DIEA (20.56 mL, 0.11 mol) was added and the solution was allowed to stir at −30° C. for 15 mins. Isobutyl chloroformate (15.44 mL, 0.11 mol) was added dropwise, taking care to adjust the addition rate so the reaction temperature remained between −35° C. and −30° C. The solution was stirred for an additional 15 mins after the addition was complete, at which time a second portion of DIEA (27.75 mL, 0.16 mol) was added. NH(OMe)Me.HCl (15.54 g, 0.16 mol) and DMF (20 mL) were then added and the reaction was allowed to warm to r.t. and stir for 1 h. The reaction was then concentrated under vacuum, and the crude residue was suspended with distilled water and extracted with diethyl ether (3×125 mL). The combined ether extracts were washed successively with dil. HCl, dil. NaHCO₃, brine, and finally dried with MgSO₄ and evaporated to dryness to give the crude material. The material was eluted through a 20 cm pad of silica gel with 4:1 Hex:EtOAc to produce a clear oil whose ¹H and ¹³C NMR spectra matched literature values. After drying the material thoroughly under high vacuum it was carried through without further purification.

### 2(S)-N-(Benzyloxycarbonyl)-N′-methoxy-N′-methyl-phenylalaninamide

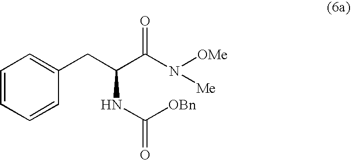

### 2(S)-N-(Isopropoxycarbonyl)-N′-methoxy-N′-methyl-phenylalaninamide

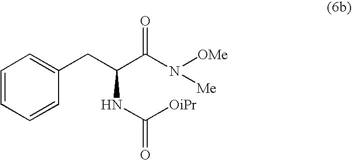

### 2(S)-N-(Ethoxycarbonyl)-N′-methoxy-N′-methyl-phenylalaninamide

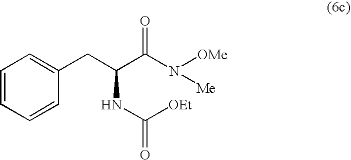

### Step 2—Perfluoroalkyl Addition To Form (Perfluoroalkyl)ketone And (Perfluoroalkyl)alcohol

A (perfluoroalkyl)ketone (7) and/or (perfluoroalkyl)alcohol (8) are synthesized from the previously-prepared N-carbamoyl esters or Weinreb amides (4) by addition of either a perfluoroalkyl Grignard reagent or a perfluoroalkyl lithium reagent as described below with representative examples.

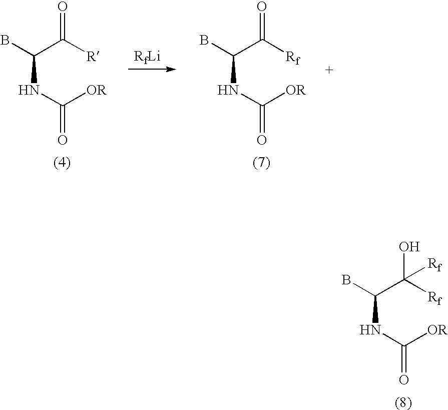

### General Procedure For the Formation And Addition of the Perfluoroalkyl Grignard Reagent

Magnesium powder (0.1 g, 4.22 mmol) was placed into a flask and flame-dried under vacuum. The flask was cooled under vacuum and then purged with nitrogen. The magnesium powder was covered with ether (2 ml) and then 1H,1H,2H,2H-perfluorooctyl iodide (0.05 g, 0.211 mmol) was added, and the suspension was stirred vigorously for 1 h. After this time the solution had become slightly grey in color while the magnesium appeared black. At this point a solution of the remaining 1H,1H,2H,2H-perfluorooctyl iodide (0.948 mL, 4 mmol) was added dropwise over 30 minutes. After the addition was completed the solution was dark grey in color characteristic of a Grignard reagent. In a separate flask the appropriate ester, 5a-c, or Weinreb amide, 6a-c, (2.11 mmol) was dissolved in dry ethyl ether (50 mL) and added dropwise to the Grignard reagent, during which a fine grey precipitate was formed. After completion of the addition the solution was allowed to stir for 4 hrs, before quenching the reaction with dil. NH₄Cl (10 mL). The solution was then extracted with ethyl ether (2×50 mL) and combined ether extracts were washed with brine, dried with MgSO₄, and evaporated to dryness. The crude material was dissolved in a minimum of ethyl ether and applied to a pad of fluorous-modified silica gel. The fluorous column was then washed with 150 mL of 7:3 MeOH:H₂O removing any organic and inorganic by-products. The (perfluoroalkyl)ketone, 9a-c, was then selectively eluted by washing the fluorous column with 250 mL of 9:1 MeOH:H₂O. Finally the bis(perfluoroalkyl)tertiary alcohol, 10a-c, was recovered by eluting with MeOH (˜100 mL).

### General Procedure For the Formation And Addition of the Perfluoroalkyl Lithium Reagent To An Ester Or Weinreb Amide

1H,1H,2H,2H-Perfluorooctyl iodide (16.9 g, 0.036 mol) was dissolved in ether:hexane to produce a solution between 0.2M and 0.5M, and cooled to −78° C. t-BuLi (20.98 mL of a 1.7M solution in pentane, 0.036 mol) was added dropwise via cannula, taking care to keep the reaction solution below −60° C. during the generation of the perfluoroalkyl lithium. In a separate flask the appropriate ester, 5a-c, or Weinreb amide, 6a-c, (0.024 mol) was dissolved in dry ethyl ether (100 mL). The electrophile solution was then added via cannula to the perfluoroalkyl lithium reagent, again adjusting the addition rate to keep the reaction solution below −60° C. The reaction was then stirred at −78° C. for about 15 minutes and then allowed to slowly warm to r.t over 2 hrs and was quenched at that time with dilute NH₄Cl (40 mL). The aqueous layer was then extracted with ethyl ether (2×50 mL). The combined ether extracts were washed with brine, dried with MgSO₄, and evaporated to dryness. The crude material was dissolved in a minimum of ethyl ether and applied to a pad of fluorous-modified silica gel. The fluorous column was then washed with 150 mL of 7:3 MeOH:H₂O removing any organic and inorganic by-products. The (perfluoroalkyl)ketone, 9a-c, was then selectively eluted by washing the fluorous column with 250 mL of 9:1 MeOH:H₂O.

### General Procedure For the Application of A Sacrificial Base In the Addition of the Perfluoroalkyl Lithium Reagent To An Ester Or Weinreb Amide

1H,1H,2H,2H-Perfluorooctyl iodide (16.9 g, 0.036 mol) was dissolved in ether:hexane to produce a solution between 0.2M and 0.5M, and cooled to −78° C. t-BuLi (20.98 mL of a 1.7M solution in pentane, 0.036 mol) was added dropwise via cannula, taking care to keep the reaction solution below −60° C. during the generation of the perfluoroalkyl lithium. In a separate flask the appropriate ester, 5a-c, or Weinreb amide, 6a-c, (0.024 mol) was dissolved in dry ethyl ether (100 mL) and cooled to −78° C. The sacrificial base (0.024 mol) was added at this point dropwise, again taking care to prevent the reaction solution from warming beyond −60° C. The cooled and deprotonated electrophile solution was then added via cannula to the perfluoroalkyl lithium reagent. The reaction was stirred at −78° C. for about 15 minutes and then allowed to slowly warm to r.t over 2 hrs and was quenched at that time with dilute NH₄Cl (40 mL). The aqueous layer was then extracted with ethyl ether (2×50 mL). The combined ether extracts were washed with brine, dried with MgSO₄, and evaporated to dryness. The crude material was dissolved in a minimum of ethyl ether and applied to a pad of fluorous-modified silica gel. The fluorous column was then washed with 150 mL of 7:3 MeOH:H₂O removing any organic and inorganic by-products. The (perfluoroalkyl)ketone, 9a-c, was then selectively eluted by washing the fluorous column with 250 mL of 9:1 MeOH:H₂O.

The following representative perfluoroketones and perfluoroalcohols were synthesized in accordance with the foregoing methods:

### (Perfluoroalkyl)ketones

### 2(S)-(Benzyloxycarbonylamino)-6,6,7,7,8,8,9,9,10,10,11,11,11-tridecafluoro-1-phenyl-undecan-3-one

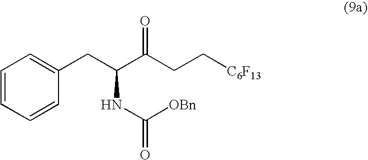

### 2(S)-(Isopropoxycarbonylamino)-6,6,7,7,8,8,9,9,10,10,11,11,11-tridecafluoro-1-phenyl-undecan-3-one

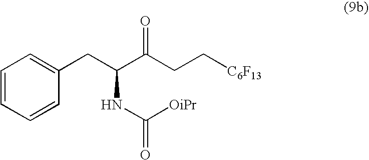

### 2(S)-(Ethoxycarbonylamino)-6,6,7,7,8,8,9,9,10,10,11,11,11-tridecafluoro-1-phenyl-undecan-3-one

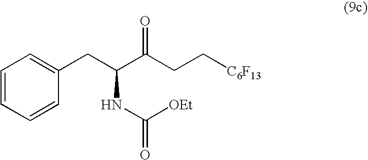

### bis(Perfluoroalkyl)alcohols

### 2(S)-(Benzyloxycarbonylamino)-3,3-bis(1′H,1′H,2′H,2′H-perfluorooctyl)-1-phenyl-propan-3-ol

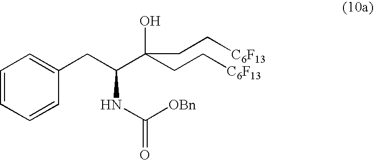

### 2(S)-(Isopropoxycarbonylamino)-3,3-bis(1′H,1′H,2′H,2′H-perfluorooctyl)-1-phenyl-propan-3-ol

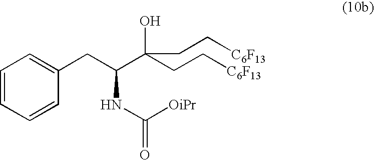

### 2(S)-(Ethoxycarbonylamino)-3,3-bis(1′H,1′H,2′H,2′H-perfluorooctyl)-1-phenyl-propan-3-ol

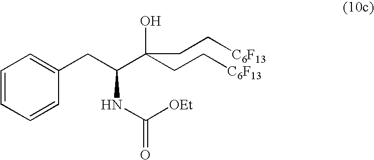

### Step 3—Diastereoselective Reduction of (Perfluoroalkyl)ketone To Protected Amino Alcohol Having the Anti Configuration

(Perfluoroalkyl)ketones were reduced to N-protected alcohols under reaction conditions that favour the anti isomer, (11), at 99:1 ratio over the syn isomer, (12).

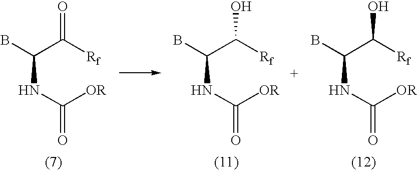

The general procedure for these diastereoselective reactions of the representative (perfluoroalkyl)ketone compounds 9a-6c is as follows:

The appropriate perfluoroketone, 9a-c, (0.017 mol) was dissolved in EtOH (100 mL) and cooled to −78° C. Powdered LiAlH(OtBu)₃ (13.44 g, 0.053 mol) was added to the flask and the reaction was allowed to warm slowly to room temperature over 16 hrs. After this time 1M HCl (30 mL) was added to the reaction. The reaction mixture was concentrated under vacuum, and the residue was resuspended with water. The aqueous mixture was extracted with ethyl ether (3×50 mL), and the combined organic fractions were dried with MgSO₄ and evaporated to dryness to give a white solid. The ratio of the two diastereomeric alcohols was established using ¹H NMR.

### (2S,3R)-(Benzyloxycarbonylamino)-6,6,7,7,8,8,9,9,10,10,11,11,11-tridecafluoro-1-phenyl-undecan-3-ol

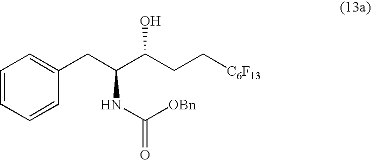

### (2S,3S)-(Benzyloxycarbonylamino)-6,6,7,7,8,8,9,9,10,10,11,11,11-tridecafluoro-1-phenyl-undecan-3-ol

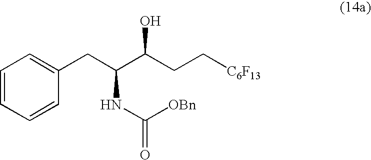

### (2S,3R)-(lsopropoxycarbonylamino)-6,6,7,7,8,8,9,9,10,10,11,11,11-tridecafluoro-1-phenyl-undecan-3-ol

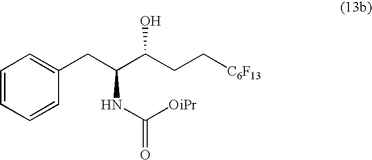

### (2S,3S)-(Isopropoxycarbonylamino)-6,6,7,7,8,8,9,9,10,10,11,11,11-tridecafluoro-1-phenyl-undecan-3-ol, 10b

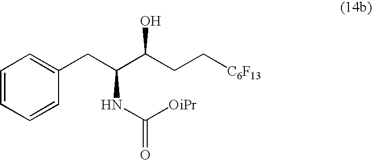

### (2S,3R)-(Ethoxycarbonylamino)-6,6,7,7,8,8,9,9,10,10,11,11,11-tridecafluoro-1-phenyl-undecan-3-ol

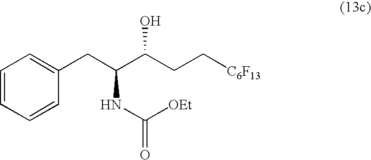

### (2S,3S)-(Ethoxycarbonylamino)-6,6,7,7,8,8,9,9,10,10,11,11,11-tridecafluoro-1-phenyl-undecan-3-ol

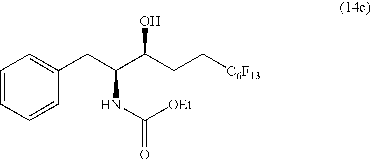

### Step 4—Cyclization To Form Oxazolidinones

By treating the anti configured (perfluoroalkyl)alcohol (11), or bis(perfluoroalkyl) tertiary alcohol (8) under alkaline conditions the material could be cyclized to form the corresponding mono-substituted (15) or di-substituted (16) oxazolidinones.

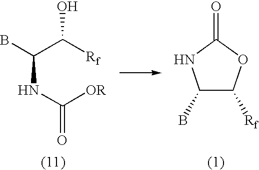

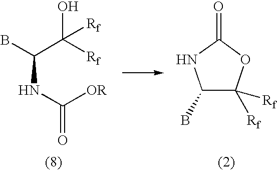

The general procedure for these cyclization reactions, involving the representative (perfluoroalkyl)alcohol 13a-c, or bis(perfluoroalkyl) tertiary alcohol 10a-c, is as follows:

The appropriate anti configured (perfluoroalkyl)alcohol, 13a-c, or bis(perfluoroalkyl) tertiary alcohol, 10a-c, (0.010 mol) was dissolved in dry THF, and NaH (2.1 g, 60% suspension in oil, 0.018 mol) was added. The slurry was stirred at r.t. for 18 hrs, and monitored by TLC. After conversion of the starting material was confirmed the reaction was quenched with 1M HCl (20 mL) and stirred rapidly until no further gas was evolved. The solution was evaporated to under vacuum dryness to yield a crude coloured residue. The crude material was dissolved in a minimum of ethyl ether and applied to a pad of fluorous-modified silica gel. The fluorous column was then washed with 200 mL of 7:3 MeOH:H₂O to remove any organic and inorganic by-products. The desired oxazolidinones, 15 and 16, were then liberated by washing the fluorous column with 9:1 MeOH:H₂O. In the case of the monosubstituted oxazolidinone 17 the relative stereochemistry of the 4 and 5 centres was assigned using 1 D selective NOE.

### (4S,5R)-4-Benzyl-5-(1′H,1′H,2′H,2′H-perfluorooctyl)-oxazolidin-2-one

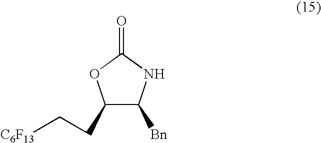

### 4(S)-Benzyl-5,5-bis(1′H,1′H,2′H,2′H-perfluorooctyl)-oxazolidin-2-one

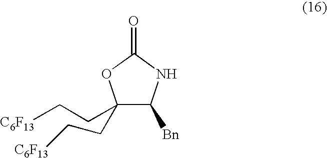

### Discussion of Experimental

In initial experiments, the perfluoroalkyl lithium species was generated in situ by adding methyl lithium to C₈F₁₇I in the presence of the electrophile.^{17, 18, 19} This procedure was important due to the instability of the anionic perfluoroalkyl reagent, even under cryogenic conditions. While this reaction can be successful it requires stringent control of addition rate, temperature and reagent concentration. Most notably, this approach suffered from significant coupling of the perfluoroalkyl lithium species with the iodide starting material. This side reaction accounted for the loss of up to 31% of the fluorous iodide, making it necessary to employ a large excess of this starting material.

The utilization of a perfluoroalkyl species containing an ethylene spacer, such as (CH₂)₂C₆F₁₃I, dramatically changes the electronic character and reactivity of the resulting nucleophile, making its behavior more akin to that of typical organometallic reagents.²⁰ This permits the application of the perfluoroalkyl nucleophile in more common protocols, allowing for the pregeneration of the reactive anionic species. The reactivity of this new nucleophile was examined with the series of known N-protected amino esters 5a-c, which were synthesized via standard literature procedures.^{14, 15, 16} The perfluoroalkyl Grignard and lithium species were generated and added to these esters, yielding the corresponding (perfluoroalkyl)ketone, 9a-c, and bis(perfluoroalkyl)tertiary alcohol, 10a-c (Table 1). Due to the relatively high electrophilicity of the (perfluoroalkyl)ketone tert-butyllithium was preferred for the generation of (perfluoroalkyl)lithium species over methyllithium or n-butyllithium.

TABLE 1
Perfluoroalkyl addition to N-protected amino esters

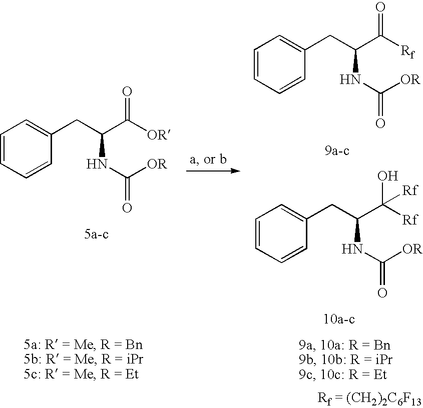

| | | | R_{f}I | | Yield | Yield |
|---|---|---|---|---|---|---|
| entry | Ester | Rxn | Eqv | Products | 9^{c} (%) | 10^{c} (%) |
| 1 | 5a | ^{a} | 1.5 | 9a:10a | 10 | 3 |
| 2 | 5a | ^{b} | 2.0 | 9a:10a | 10 | 0 |
| 3 | 5b | ^{a} | 3.0 | 9b:10b | 30 | 17 |
| 4 | 5b | ^{b} | 1.5 | 9b:10b | 39 | 19 |
| 5 | 5b | ^{b} | 2.0 | 9b:10b | 47 | 32 |
| 6 | 5b | ^{b} | 3.0 | 9b:10b | 12 | 68 |
| 7 | 5c | ^{a} | 3.0 | 9c:10c | 42 | 36 |
| 8 | 5c | ^{b} | 2.0 | 9c:10c | 18 | 2 |

Key:
^{a} Mg, (CH₂)₂C₆F₁₃I, Ether;
^{b} tBuLi, (CH₂)₂C₆F₁₃I, Ether:Hex, −78° C. - r.t.;
^{c} Yield represent % conversion from corresponding electrophile.

Comparing the two methods, the lithium protocol was found to be more reproducible, and operationally easier. Using the Grignard protocol acceptable levels of perfluoroalkyl addition were only observed when a three-fold excess (entry 3) of the fluorous iodide was employed. In contrast, the lithium protocol provided good yields of fluorous materials with only a slight excess of the required iodide (entry 4). The only exception to this trend was seen with perfluoroalkyl addition to ethyl carbamate-protected ester 5c (entry 7 vs 8). This result was attributed to solubility problems with 5c under the cryogenic conditions necessary for the lithium addition, which prevented efficient incorporation. Overall, the isopropyl carbamate-protected ester 5b performed best, allowing the isolation of ketone 9b and tertiary alcohol 10b in good yields.

The relative ratio of these two materials could be controlled to some extent, allowing primarily the tertiary alcohol to be generated when an excess of the perfluoroalkyl species was employed (entry 6). However, it was not possible to select for the ketone, even when only a stoichiometric equivalent of the perfluoroalkyl iodide was used.

To obtain the ketone selectively it was necessary to use the corresponding carbamate-protected Weinreb amides 6a-c, (Table 2), which were synthesized from the carbamate-protected amino acids using modifications of reported procedures.^{21, 22} The published mixed anhydride methods often lead to significant levels of epimerization and thus the generation of racemic products. This was not significantly improved by employing other coupling agents, such as CDMT²³ or EDCl²⁴. We established that the epimerization could be completely suppressed by stringent temperature control during the generation of the mixed anhydride.

TABLE 2
Perfluoroalkyl addition to N-protected Weinreb amides

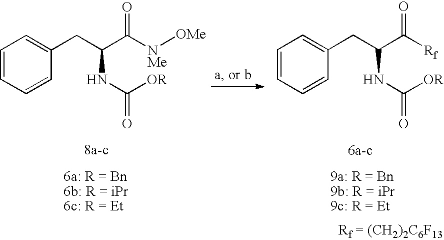

| | | | R_{f}I | | |
|---|---|---|---|---|---|
| entry | Ester | Rxn | Eqv | Product | Yield |
| 1 | 6a | ^{a} | 2.5 | 9a | 27% |
| 2 | 6a | ^{b} | 1.5 | 9a | 30% |
| 3 | 6b | ^{a} | 3.0 | 9b | 18% |
| 4 | 6b | ^{b} | 2.3 | 9b | 38% |
| 5 | 6c | ^{a} | 2.5 | 9c | 17% |
| 6 | 6c | ^{b} | 1.5 | 9c | 48% |
| 7 | 6c | ^{b, c} | 1.5 | 9c | 68% |
| 8 | 6c | ^{b, d} | 1.5 | 9c | 75% |

^{a} Mg, (CH₂)₂C₆F₁₃I, Ether, −15° C.;
^{b} tBuLi, (CH₂)₂C₆F₁₃I, Ether:Hex, −78° C. - r.t.;
^{c} n-BuLi used as sacrificial base;
^{d} t-BuLi used as sacrificial base.

The perfluoroalkyl lithium was again found to be superior to the Grignard method in this series. The trend in the reactivity of the Weinreb amides also mirrors that of the esters, with higher reactivity found in the electrophiles bearing smaller carbamate protecting groups. It is possible that this trend reflects a reduction in acidity of the carbamate nitrogen as the size of the carbamate alkyl group decreases.

Recent studies have shown that nucleophilic addition to Weinreb amides can be improved by employing sacrificial Grignard or alkyl lithium bases.^{25, 26} By treating the Weinreb amide with n-butyl lithium or t-butyl lithium (entry 7 and 8) it was possible to increase the yield of perfluoroketone 9c to 68% and 75% respectively, while allowing a reduction in the amount of perfluoroalkyl iodide required.

Ketones 9a-c could be selectively reduced to the anti configured alcohols 13a-c (Table 3) using chelation control, as demonstrated by Hoffmann et al.²⁷ However, high diastereoselectivity was not observed with completely fluorinated ketones 9d and 9e (entry 5 to 7).¹⁷ This is likely due to the reduced Lewis basicity of the carbonyl resulting from the strong electron withdrawing effects of the C₈F₁₇ group. The ethylene spacer in ketones 9a-c is therefore necessary and crucial to provide high diastereoselectivity by insulating the carbonyl. This feature makes it possible to give tight coordination to the amide and leads to selective reduction via chelation control. It was also noted that the nature of the alkyl group of the carbamate had no effect on the selectivity of the reaction, indicating that the stereochemical outcome was dominated by the benzyl substituent of the amino ketones.

TABLE 3
Diastereoselective reduction via chelation control

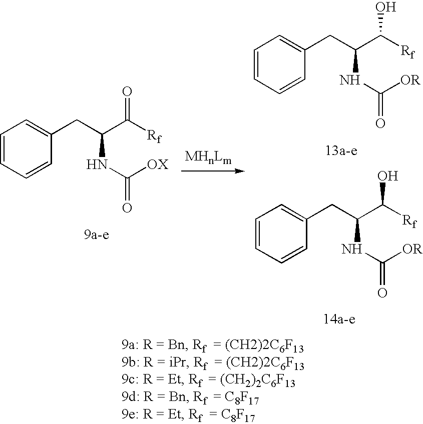

| | | | | Selectivity^{a} |
|---|---|---|---|---|
| entry | Ketone | Method | Yield | (13:14) |
| 1 | 9a | A | 98% | 4:1 |
| 2 | 9a | B | 98% | >99:1 |
| 3 | 9b | B | 95% | >99:1 |
| 4 | 9c | B | 99% | >99:1 |
| 5 | 9d | B | 97% | 4:1 |
| 6 | 9e | A | 97% | 3:1^{b} |
| 7 | 9e | B | 99% | 5:1^{b} |

^{a}Selectivity determined using ¹H NMR;
^{b}results from reference¹⁷;
Reduction method: A, NaBH₄, EtOH, −78° C. to r.t.; B, LiAlH(OtBu)₃, EtOH, −78° C. to r.t.

Alcohols 13a-c and 10a-c were cyclized with NaH in THF to afford oxazolidinones 17 and 18 respectively. In the optimal embodiment it is possible to obtain oxazolidinone 15 through five chemical transformations in 60% overall yield, and oxazolidinone 16 through four chemical transformations in 47% overall yield.

An important aspect of this strategy was the application of fluorous solid-phase extraction (FSPE). After the perfluoroalkyl fragment had been introduced into the molecule all subsequent compounds were easily purified using this technique where necessary. Reactions were simply quenched and evaporated to yield crude residues. The material was applied to the fluorous silica gel in a concentrated solution and eluted with a fluorophobic solvent (70:30 MeOH:H₂O) to strip the organic and inorganic impurities. Switching to a more fluorophilic solvent (such as methanol or diethyl ether) allowed the desired fluorous products to be isolated. No further purifications were necessary and the materials were carried forward in the synthesis. Using this technique we were able to synthesize the oxazolidinones on scales as large as 25 g using 150 ml of fluorous-modified silica gel. The scale of the operation is limited only by the size of the column that is available for solid-phase extraction.

In conclusion, the methodologies described herein enable the syntheses of a new class of fluorous oxazolidinone chiral auxiliary in a practical manner on a large scale. Coupling the optimized synthesis with FSPE allows the material to be rapidly processed, making it possible to generate multi gram quantities of the materials rapidly and efficiently.

### Auxiliary Functionality

The general fluorous oxazolidinone auxiliary, 1, is characterized by three main functional groups that provide unique functionality in the synthesis and purification of chiral compounds in automated and parallel technologies. These functional groups include the fluorous component, the non-functional hydrocarbon component and the active N-site

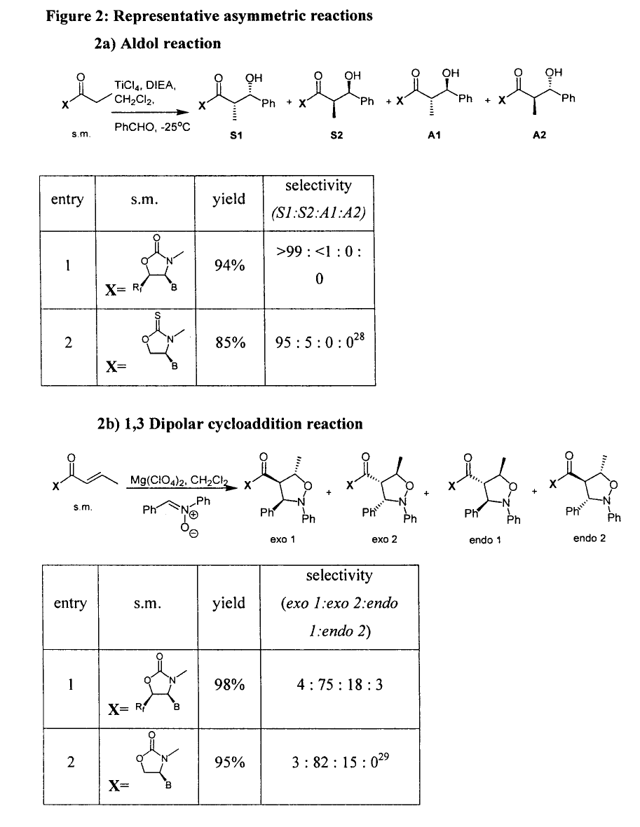

The functionality of compounds with the general structure 1 have been tested in several model aldol reactions, radical conjugate addition reactions, and 1,3 dipolar cycloaddition reactions. These reactions were carried out using standard solution phase procedures, producing yields and selectivities similar to those obtained using their non-fluorous counterparts. Moreover, the reactions could be rapidly purified by virtue of the fluorous nature of the auxiliary I and its derivatives. Examples of theses asymmetric transformations can be found in FIG. 2 along with data from the related non-fluorous chiral auxiliaries.

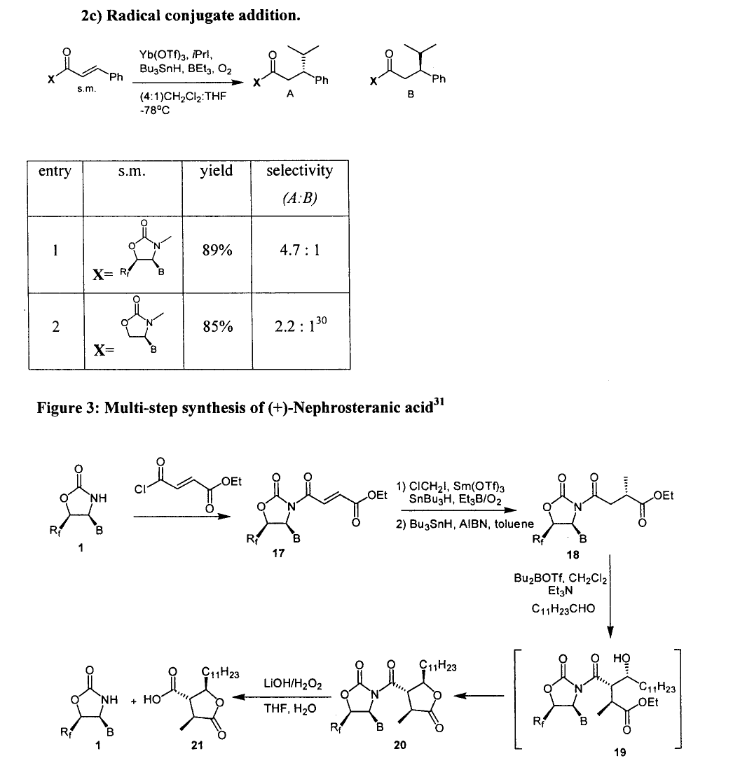

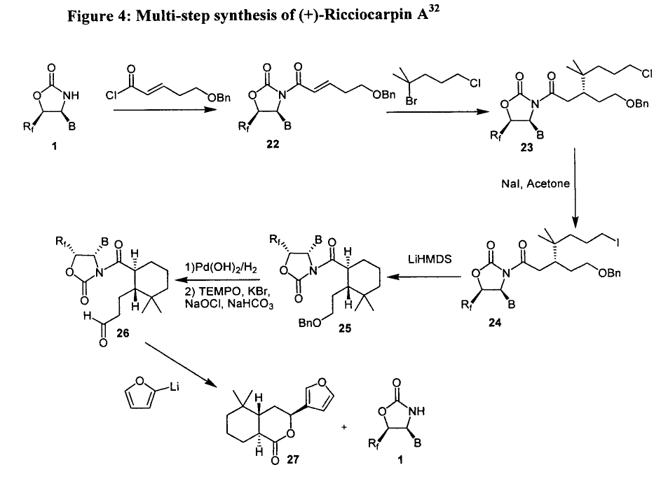

It is apparent that the robustness of these fluorous auxiliaries and ease of product recovery they permit will facilitate multi-step supported synthetic protocols. This will be useful in parallel or combinatorial chemistry, greatly assisting natural product synthesis and drug discovery. FIGS. 3 and 4 show examples of this type of application, based on established asymmetric free-radical conjugate additions.

FIG. 3 details the synthesis of (+)-Neophrosteranic acid, 21, using chemistry described by Sibi et al.³¹ Neophrosteranic acid is a member of an important class of natural product known as butyrolacetones, which show many promising biological activities, including anti-cancer and anti-HIV properties. Following formation of the asymmetric fumerate derivative 17 radical conjugate addition introduces a methyl group stereoselectively to give compound 18. This material can then be carried through an aldol reaction using a boron enolate, giving the intermediate alcohol 19, which cyclizes under reaction conditions to give lactone 20. The natural product 21 is then liberated via alkane hydrogen peroxide cleavage, allowing the fluorous auxiliary 1 to be recovered.

FIG. 4 details the synthesis of (+)-Ricciocarpin A, 27, using a modification of chemistry developed by Sibi et al.³² (+)-Ricciocarpin is a furanosesquiterpene lactone exhibiting high molluscicidal activity against the water snail Biomphalaria glabtata, which is involved in the schistosomiasis life cycle as a vector. Synthesis is achieved by functionalizing the fluorous chiral auxiliary with the pictured unsaturated acid chloride, giving compound 22. Asymmetric conjugate radical addition gives rise to the halogenated compound 23, which can be converted to the iodide, 24. This material then undergoes an intramolecular ring closure via an enolate intermediate to give the trans six-member ring in 25. Deprotection of the alcohol followed by TEMPO oxidation yields the required aldehyde 26. The synthesis is completed by adding 3-lithofuran, which adds to the aldehyde center to generate an intermediate alkoxy species. This intermediate attacks the exocyclic carbonyl to form the lactone functionality of (+)-Ricciocarpin A, 27, and subsequently displaces fluorous auxiliary 1.

These two examples demonstrate elegant chemistry which have been proven using a non-fluorous auxiliaries or chiral Lewis acids. In both cases they take advantage of the chiral oxazolidinone's ability to direct the stereochemistry in numerous established reactions, specifically the radical conjugate addition (forming compound 18 and 23), and the boron-mediated aldol reaction (forming intermediate 19), and intramolecular ring closure (forming compound 25). While these routes are efficient and have been proven using traditional chiral auxiliaries and catalysts, standard chromatography and manual purification methods must be applied after each step. This has the effect of significantly adding to the work necessary to complete the task, and limiting the ability to vary the reactions.

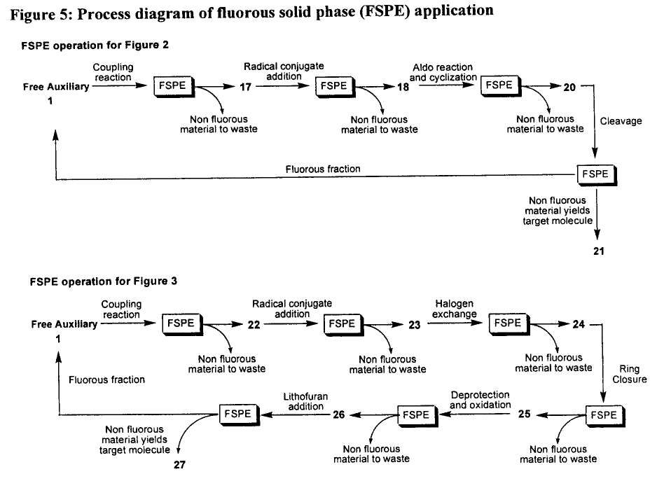

By applying the perfluoroalkyl auxiliary 1 each material would be rapidly purified by virtue of its inherent fluorous nature simply using fluorous solid phase extraction (FSPE). An operational schematic of this in action is pictured in FIG. 5. In each case, the material is purified via FSPE following reaction completion, greatly reducing the time and effort required to isolate the target compounds. Furthermore, a parallel library of related materials could easily be generated through slight variations in the nature of the starting materials, without significantly complicating the isolation and purification process. It is this ability to offer efficient and effortless recovery of synthetic materials coupled with robust and versatile stereoselective transformations that makes the invention a truly powerful tool for drug discovery and high-throughput chemistry.

The above-described embodiments of the present invention are intended to be examples only. Alterations, modifications and variations may be effected to the particular embodiments by those of skill in the art without departing from the scope of the invention, which is defined solely by the claims appended hereto.

### REFERENCES

¹ Kirchho., J. H.; Lormann, M. E. P.; Brase, S. Chim. Oggi. 2001, 19, 28.
² Chung, C. W. Y.; Toy, P. H. Tet. Asymm. 2004, 15, 387.
³ Purandare, A. V.; Natarajan, S., Tet. Lett., 1997, 38, 8777.
⁴ Phoon, C. W.; Abell, C. Tet. Lett., 1998, 39, 2655.
⁵ Burgess, K.; Lim, D. J. Chem. Soc, Chem. Commun., 1997, 785.
⁶ Studer, A.; Jeger, P.; Wipf, P.; Curran, D. P. J. Org Chem. 1997, 62, 2917.
⁷ Curran, D. P.; Hadida, S.; He, M. J. Org. Chem. 1997, 62, 6714.
⁸ Nakamura, Y.; Takeuchi, S.; Okumura, K.; Ohgo, Y.; Curran, D. P. Tetrahedron 2002, 58, 3963.
⁹ Dandapani, S.; Curran, D. P. Tetrahedron 2002, 58, 3855.
¹⁰ Zhang, W. Tetrahedron 2003, 59, 4475.
¹¹ Ager, David J.; Prakash, Indra; Schaad, David R. Aldrichimica Acta 1997, 30, 3.
¹² Kruse, C. H.; Holden, K. G. J. Org. Chem. 1985, 50, 2792.
¹³ Edmonds, M. K.; Abell, A. D. J. Org. Chem., 2001, 66, 3747.
¹⁴ You, J.; Wróblewski, A. E.; Verkade, J. G. Tetrahedron 2004, 60, 7877.
¹⁵ Matsuda, F.; Matsumoto, T.; Ohsaki, M.; Ito, Y.; Terashima, S. Bull. Chem. Soc. Jpn. 1992, 65, 360.
¹⁶ Lewis, N.; McKillop, A.; Taylor, R. J. K.; Watson, R. J. Synth. Comm. 1995, 25, 561.
¹⁷ Hein, J. E.; Hultin, P. G. Synlett 2003, 635.
¹⁸ Gassman, P. G.; O'Reilly, N. J. Tet. Lett. 1985, 26, 5243.
¹⁹ Uno, H.; Shiraishi, Y.; Suzuki, H. Bull. Chem. Soc. Jpn. 1989, 62, 2636.
²⁰ Legros, J.; Crousse, B.; Bonnet-Delpon, D.; Begue, J.-P. Tetrahedron 2002, 58, 3993.
²¹ Cupps, T. L.; Boutin, R. H.; Rapoport, H. J. Org. Chem. 1985, 50, 3972.
²² Angelastro, M. R.; Peet, N. P.; Bey, P. J. Org. Chem. 1989, 54, 3913.
²³ Luca, L. D.; Giacomelli, G.; Taddei, M. J. Org. Chem. 2001, 66, 2534.
²⁴ Collier, P. N.; Campbell, A. D.; Patel, I.; Raynham, T. M.; Taylor, R. J. K. J. Org Chem, 2002, 67, 1802.
²⁵ Liu, J.; Ikemoto, N.; Petrillo, D.; Armstrong, J. D., III Tet. Lett. 2002, 43, 8223.
²⁶ So, R. C.; Ndonye, R.; Izmirian, D. P.; Richardson, S. K.; Guerrera, R. L.; Howell, A. R. J. Org. Chem. 2004, 69, 3233.
²⁷ Hoffman, R. V.; Maslouh, N.; Cervantes-Lee, F. J. Org. Chem. 2002, 67, 1045.
²⁸ Crimmins, M. T.; King, B. W.; Tabet, E. A., Chaudhary, K. J. Org. Chem., 2001, 66, 894.
²⁹ Desimoni, G.; Faita, G.; Mella, M.; Righetti, P.; Zema, M. Tetrahedron 1999, 55, 8509.
³⁰ Sibi, M. P.; Ji, J.; Sausker, J. B.; Jasperse, C. P. J Am. Chem. Soc. 1999, 121, 7517.
³¹ Sibi, M. P.; Lui, P.; Ji, J.; Hajra, S.; Chen J. J. Org. Chem. 2002, 67, 1738.
³² Sibi, M. P.; He, L. Org. Lett. 2004, 6, 1749.

1. A compound of the formula 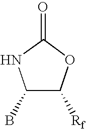
wherein R_{f} is a perfluroalkyl group having the general formula (CH2)ₓ—C_{y}F_{2y+1} where x=1-5 and y=4-10 and wherein B is an unfunctionalized aryl, arylaykyl, or alkyl group.

2. A compound of the formula 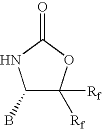
wherein R_{f} is a perfluroalkyl group having the general formula (CH2)ₓ—C_{y}F_{2y+1} where x=1-5 and y=4-10 and wherein B is an unfunctionalized aryl, arylaykyl, or alkyl group.

3. A compound as in claim 1 wherein the compound is enantomerically pure.

4. A compound as in claim 1 wherein x=2 and y=6.

5. A compound as in claim 1 wherein B is derived from unfunctionalized amino acids.

6. A method of preparing the compound of claim 1 comprising the steps of:
a. synthesizing an N-carbamate protected acyl species from an unfunctionalized amino acid;
b. subjecting the N-carbamate protected acyl species to a perfluoroalkyl lithium addition reaction using a perfluoroalkyllithium species to form a (perfluoroalkyl)ketone;
c. subjecting the (perfluoroalkyl)ketone to a diastereoselective reduction reaction to form a protected amino alcohol having an anti configuration;
d. subjecting the protected amino alcohol to a cyclization reaction to form the compound of claim 1.

7. A method as in claim 6 wherein the N-carbamate protected acyl species is an N-(ethoxycarbonyl) group, and B is CH₂(C₆H₅).

8. A method as in claim 6 wherein t-BuLi is used to form the perfluoroalkyllithium species, in a mixture of diethyl ether and hexanes or other hydrocarbon co-solvent at a temperature not exceeding −60 ° C.

9. A method as in claim 6 wherein the N-carbamoyl acyl species is first deprotonated using a sacrificial base.

10. A method as in claim 9 wherein the sacrificial base is selected from any one of or a combination of n-BuLi, s-BuLi, t-BuLi, NaH, KH or other alkali metal hydride.

11. A method as in claim 9 wherein the sacrificial base is t-BuLi.
